(19) United States
(12) Patent Application Publication
(10) Pub. No.: US 2003/0059349 A1
(43) Pub. Date: Mar. 27, 2003

(54) Contacting device

(76) Inventor: Andre Howe, Berlin (DE)

Correspondence Address: KRIEGSMAN & KRIEGSMAN, 665 FRANKLIN STREET, FRAMINGHAM, MA, 01702, US

(21) Appl. No.: 10/181,630

(22) PCT Filed: Jan. 17, 2001
(86) PCT No.: PCT/DE01/00236

(30) Foreign Application Priority Data
Jan. 17, 2000 (DE) 10002920.5

(51) Int. Cl. B01L 3/00; C12M 1/00

(52) U.S. Cl. 422/102; 422/99; 422/104; 435/283.1; 435/287.2; 435/288.3

(57) ABSTRACT

A device is described for the contacting of biological substances immobilized on a surface with a solution of biological substances, which [device] comprises a chamber for the uptake of the solution and is particularly suitable for the contamination-free hybridization of DNA specimens on nucleic acids or oligonucleotide probes.

The invention concerns a device for the contacting of biological substances immobilized on a surface with a solution of biological substances.

Hybridizations of specimen DNA on oligomer chips, for example, oligonucleotide arrays, are conducted for the detection of specific sequences in the specimen DNA. A possible approach “sequencing by hybridization (SBH)”, determines in fact the complete sequence of specimen DNA or at least large portions thereof. Allele-specific hybridizations, however, are also conducted in order to detect specific changes in the specimen DNA, e.g., point mutations. The present invention serves for the sequential conducting of a large number of such experiments, wherein particular value is placed on a contamination-free operation.

Hybridization chambers have been made commercially available in the meantime by different companies, but as a rule cannot be temperature-controlled separately. Hybridization chambers, which are also suitable for uptake of specimen supports or slides, are marketed, e.g., by the company GeneMachines, CA, USA and by the Telechem company.

The company Genetic Solutions has also made available pneumatically controllable hybridization chambers that can be temperature controlled, in which the hybridization properties will be improved by the movement of the hybridization fluid.

A number of hybridization chambers are known from the prior art. U.S. Pat. No. 5,100,775; U.S. Pat. No. 5,360,741 or U.S. Pat. No. 5,466,603 thus describe hybridization chambers, which are adapted for the most varied objectives and needs. Such hybridization chambers have been made available commercially in many designs, but cannot generally be separately temperature-controlled. Also, suitable hybridization chambers are known for the uptake of specimen supports or slides. In addition, foils, which can form hybridization chambers by gluing onto specimen supports or slides are available, which are self-adhering at the edges. A pneumatically controllable and temperature-controlled hybridization chamber, in which the hybridization properties will be improved by the movement of the hybridization fluid is also known.

The chambers that have been available previously, however, have either the disadvantage that they must be cleaned in a time-consuming manner between different experiments or they are completely designed for one-time use.

The device described here for contacting immobilized biological substances will be reusable as often as desired and the problem of contamination of the following experiment by components of the previous experiment will be solved in that easily exchangeable, easy-to-clean components exclusively come into contact with the solution.

In addition, the form and size of the contact surface between the immobilized specimen and the solution of biological substances can be individually selected by the configuration of the device according to the invention. Thus, an adaptation of the chamber of the device to the most varied specimen formats is possible.

The subject of the present invention is a device for the contacting of biological substances immobilized on a surface 5 with a solution of biological substances. It is comprised of a chamber for uptake of the solution, the under side of which is formed by surface 5 and the upper side of which is formed by a cover 3 and the side walls of which are formed by a gasket 6. Cover 3 can be exchanged for cleaning purposes. It contains at least two openings for introducing and/or discharging the solution. A constant pressure is exercised on gasket 6 by means of springs.

In a particularly preferred embodiment of the device, the biological substances immobilized on surface 5 are an arrangement of oligonucleotides, peptide nucleic acids, modified oligonucleotide or nucleic acids. The solution of biological substances that is used correspondingly preferably contains oligonucleotides, peptide nucleic acids, modified oligonucleotides or nucleic acids.

In a particularly preferred embodiment, cover 3 of the device is comprised of two parts, the first of which can be exchanged without additional means, contains drilled holes for introducing the solution and comprises the contact surface for the chamber as well as a groove for uptake of the gasket, and the second part serves for uptake and locking of the first part and also contains the springs that control the pressure force of the first part on the gasket. The first cover part that can be detached preferably contains recesses is and additionally preferably drilled holes, which serve for uptake of the springs of the second cover part.

Preferably, the spring force is transferred to the gasket by pressure pieces on the second cover part, which are attached via rubber rings in the above-named drilled holes of the first cover part.

In a particularly preferred embodiment of the device, the pressure force of the cover on the gasket is limited by a mechanical barrier. The chamber volume and the shape of the chamber are preferably defined by a cavity contained in the cover.

In a particularly preferred form of embodiment of the device, the chamber is configured as a rectangle.

In another preferred form of embodiment of the device, the chamber is configured as an oval.

In a particularly preferred form of the device, the chamber is configured as a hexagon.

In another particularly preferred variant of the device according to the invention, the chamber is configured as an oval with points formed on the narrow sides

In another particularly preferred variant of the device according to the invention, the chamber is configured as an oval with projections formed on the narrow side.

In another particularly preferred variant, the cover of the device contains threads for introducing pipeline connections. Preferably, flexible tubing lines are introduced on the cover for introducing the specimen or moving the specimen over the surface with.

In another particularly preferred embodiment, the chamber of the device is temperature-controlled by means of one or more Peltier elements.

In another particularly preferred embodiment, the cover of the device can be swung open and can be locked in the closed position.

In another particularly preferred embodiment, the surface 5 is comprised of glass and preferably is a glass slide, as is also used for purposes of microscopy.

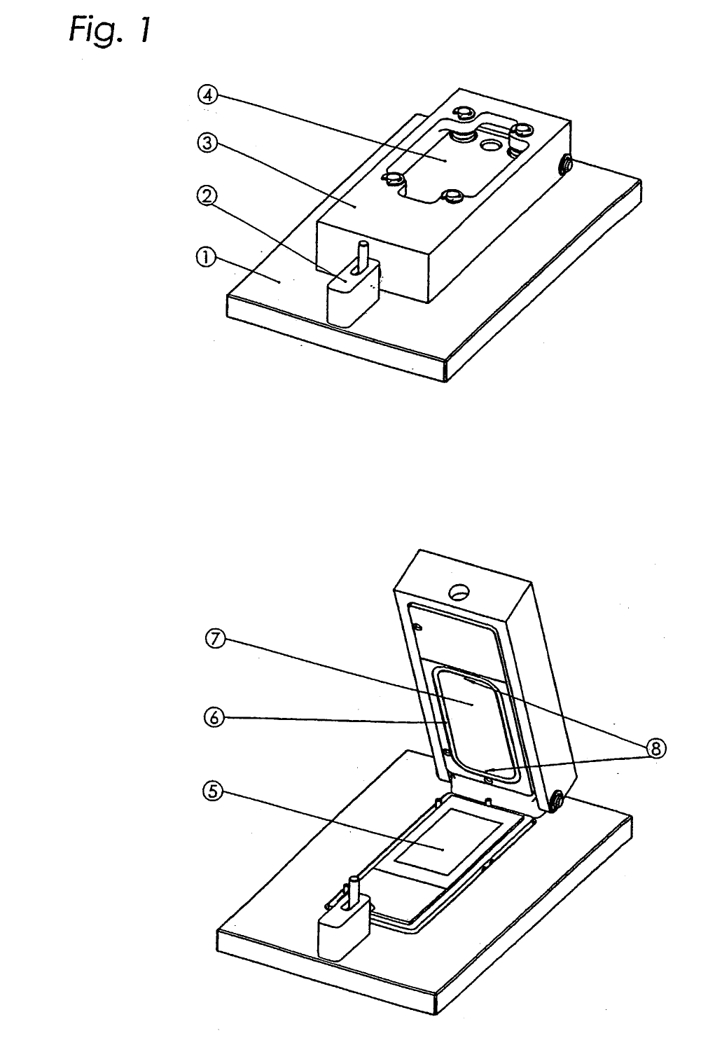

FIGS. 1 to 3 illustrate the invention.

FIG. 1 shows the device in swung-open position to view onto surface 5, gasket 6, chamber volume 7 and the inlet and outlet openings 8.

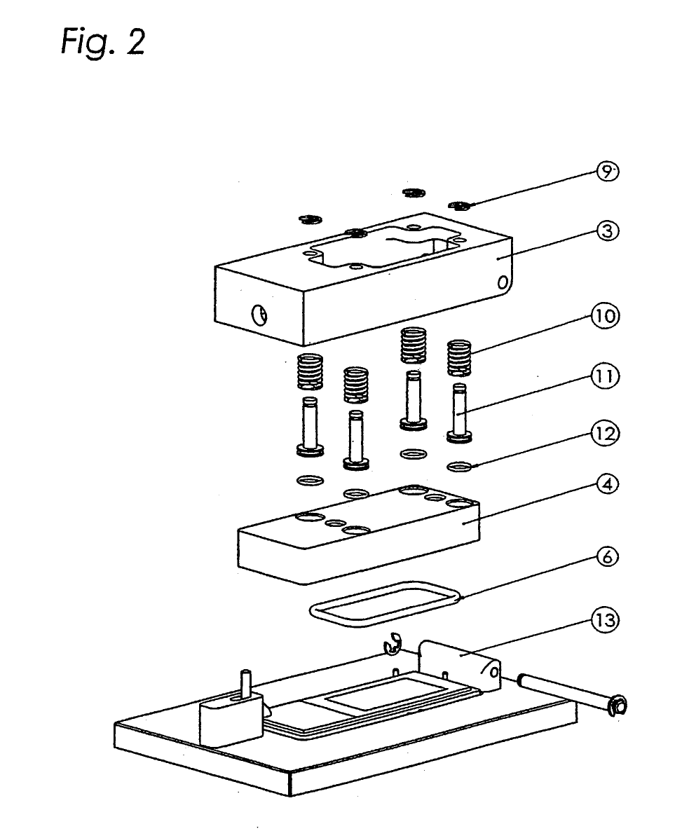

FIG. 2 shows a view of the device, in which the components of cover 3 are separately shown.

The arrangement of the tappets with the compression springs over the rubber gasket guarantees a reliably functioning, reproducible sealing of the chamber volume. The pressure force is produced by the 4 springs.

The insert is held by 4 O-rings on the tappets. It does not fall off due to its own weight, but can be removed from the cover with slight finger pressure from behind. The inserts can be easily cleaned separately after each experiment.

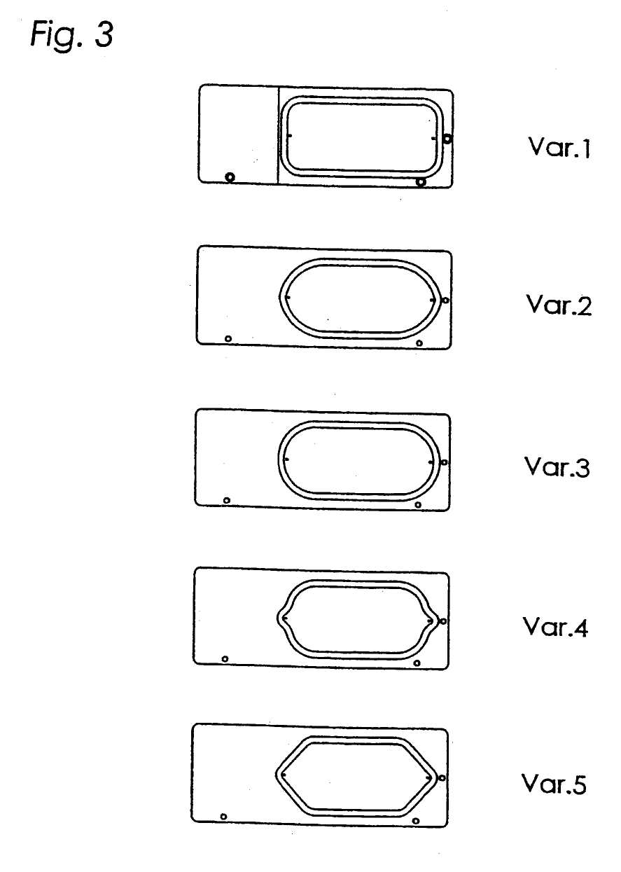

FIG. 3 shows, for example, preferred forms of embodiment of the device according to the invention, in which the chamber possesses various shapes and/or sizes. Variants 1 and 5 show rectangular and hexagonal-rectangular configurations of the chamber. Variants 2 and 3 illustrate examples of embodiment, in which the basic chamber form is oval.

### LIST OF REFERENCE SYMBOLS

| | |
|---|---|
| 1 | base plate |
| 2 | locking device |
| 3 | cover |
| 4 | chamber insert |
| 5 | surface (specimen surface) |
| 6 | gasket |
| 7 | chamber volume |
| 8 | inlet and outlet openings |
| 9 | lock washer |
| 10 | spring |
| 11 | pressure piece |
| 12 | O-ring |
| 13 | hinge |

Patent claims

1. A device for the contacting of biological substances immobilized on a surface (5) with a solution of biological substances is hereby characterized in that it comprises a chamber for uptake of the solution, the underside of which is formed by surface (5) and the upper side of which is formed by a cover (3) and whose side walls are formed by a gasket (6), whereby the cover (3) can be exchanged for cleaning purposes, it contains at least two openings for introducing the solution and exercises a constant pressure on gasket (6) by means of springs.

2. The device according to claim 1, further characterized in that the biological substances immobilized on surface (5) are an arrangement of oligonucleotides, peptide nucleic acids, modified oligonucleotides or nucleic acids.

3. The device according to claim 1 or 2, further characterized in that the solution of biological substances contains oligonucleotides, peptide nucleic acids, modified oligonucleotides or nucleic acids.

4. The device according to one of the preceding claims, further characterized in that cover (3) is comprised of two parts, the first of which can be exchanged without additional means, contains drilled holes for introducing the solution and comprises the contact surfaces for the chamber as well as a groove for uptake of the gasket, and the second part serves for uptake and locking of the first part and also contains the springs which regulate the pressure force of the first part on the gasket.

5. The device according to claim 4, further characterized in that the first, removable cover part contains recesses and preferably drilled holes, which serve for the uptake of the springs of the second cover part.

6. The device according to claim 5, further characterized in that the pressure pieces on the second cover part by means of which the spring force is transferred to the gasket, are attached via rubber rings in the drilled holes of the first cover part.

7. The device according to one of the preceding claims, further characterized in that the pressure force of the cover on the gasket is limited by a mechanical barrier.

8. The device according to one of the preceding claims, further characterized in that the cover contains a cavity, which defines the chamber volume and the shape of the chamber.

9. The device according to one of the preceding claims, further characterized in that the cover contains threads for introducing pipeline connections.

10. The device according to one of the preceding claims, further characterized in that flexible lines are introduced on the cover for introducing and/or for moving the specimen over the surface.

11. The device according to one of the preceding claims, further characterized in that the chamber can be temperature-controlled by means of one or more Peltier elements.

12. The device according to one of the preceding claims, further characterized in that the cover can be swung open and can be locked in the closed position.

13. The device according to one of the preceding claims, further characterized in that surface (5) is comprised of glass and is preferably a glass slide, as is also used for microscopy purposes.

15. The device according to claim 8, further characterized in that the chamber is configured as a rectangle.

16. The device according to claim 8, further characterized in that the chamber is configured as an oval.

17. The device according to claim 8, further characterized in that the chamber is configured as a hexagon.

18. The device according to claim 8, further characterized in that the chamber is configured as an oval and with points formed on the narrow sides.

19. The device according to claim 8, further characterized in that the chamber is configured as an oval and with projections formed on the narrow sides.
